(19) United States
(12) Patent Application Publication
(10) Pub. No.: US 2014/0348958 A1
(43) Pub. Date: Nov. 27, 2014

(54) Anticancer Active Ingredient From Guiera Senegalensis

(71) Applicants: UNIVERSITE D'AUVERGNE CLERMONT I, CLERMONT FERRAND (FR); Université Blaise Pascal - CLERMONT II, CLERMONT-FERRAND CEDEX 1 (FR); ECOLE NATIONALE SUPERIEURE DE CHIMIE DE CLERMONT FERRAND, AUBIERE CEDEX (FR)

(72) Inventors: Pierre Chalard, Chanonat (FR); Florence Caldefie-Chezet, Issoire (FR); Laetitia Delort, Clermont-Ferrand (FR); Kirti Patel, Clermont-Ferrant (FR)

(21) Appl. No.: 14/345,051

(22) PCT Filed: Sep. 12, 2012
(86) PCT No.: PCT/EP2012/068125

§ 371 Date: Mar. 14, 2014

(30) Foreign Application Priority Data
Sep. 16, 2011 (FR) 1158256

(51) Int. Cl. C07C 49/753; A61K 45/06; A61K 36/185; A61K 31/122

(52) U.S. Cl. CPC C07C 49/753; A61K 31/122; A61K 45/06; A61K 36/185
USPC 424/725; 568/374; 514/691; 514/651; 514/274; 514/283; 514/449

(57) ABSTRACT

A compound of formula (I), a positional or structural isomer thereof, or a pharmaceutically acceptable base addition salt or acid addition salt thereof, use thereof as a medicament, in particular an anticancer medicament.

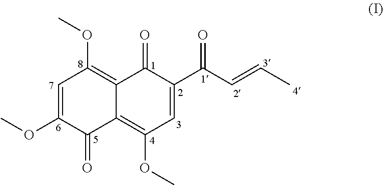 [SMILES: C/C=C/C(=O)C1=CC(OC)=C2C(=O)C(OC)=CC(OC)=C2C1=O]

The present invention relates to a novel compound which may be used as a pharmaceutical active ingredient, notably in the treatment of cancer. This compound may appear as a concentrated plant extract or as a synthetic compound.

Guiera senegalensis is a very common plant in tropical Africa. As a sudano-sahelian species, its areas of abundance are mainly in Western Africa. This plant is very common in the Sahel where it forms mono-specific populations. It abounds in fallows on clayey or sandy soils, is locally gregarious, and also exists in silts of temporarily flooded valleys. It is present in almost the whole of Mali, over an area extending from Kayes to Gossi (Timbuktu region). An invasive species of sandy fallow lands, first colonizer of lands burnt by bush fires in Sudanese and Sudano-guinean savannahs, an element of degraded savannahs, it is also a pioneering plant disseminated by cattle in the fallows, and is also an indicator of overgrazing. The geographical area of the Guiera and its areas of abundance have mainly been established in Western Africa. Its area extends parallel to the Equator from Senegal to the frontiers of Ethiopia.

TABLE 1
Taxonomy,

| | |
|---|---|
| Kingdom | Plant |
| Sub-kingdoms | Eukaryots |
| Branch | Spermaphytes |
| Sub-branch | Angiosperms |
| Class | Dicotyledones |
| Sub-class | Rosidae |
| Order | Myrtales |
| Family | Combretaceae |
| Genus | Guiera |
| Species | senegalensis |

Guiera senegalensis is mainly recognized for its anti-inflammatory, antiseptic, anti-tussive, diuretic, eupneic and febrifuge properties, whence its prescription for coughs, dyspneic conditions, malaria, pneumopathies, bronchopathies. These are leaves which are generally used as a decoction or maceration in a bath or as a drink. In external use, Guiera senegalensis is considered as an antiseptic healing vulnerary plant for treating wounds, stomatitises, gingivitises, syphilitic chancres (Kerharo J. et Adams (1974), La Pharmacopée sénégalaise Traditionnelle. Plantes Médicinales et Toxiques. Imprimerie JOUVE, 17 rue du LOUVRE, 75001 PARIS. 354).

A methoxylated naphthyl butenone, called Guieranone A was isolated from the leaves of Guiera senegalensis (Olga Silva and Elsa T. Gomes. (2003), Guieranone A, a Naphthyl Butenone from the Leaves of Guiera senegalensis with Antifungal Activity. J. Nat. Prod., 66, 447).

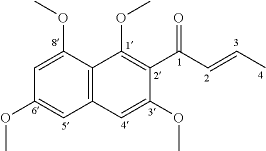 [SMILES: C/C=C/C(=O)c1c(OC)cc2cc(OC)cc(OC)c2c1OC]

### Guieranone A

The inventors were able to isolate a novel compound from Guiera senegalensis. This novel compound called Guieranone B has the following formula (I).

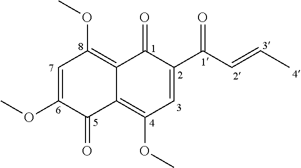 [SMILES: C/C=C/C(=O)C1=CC(OC)=C2C(=O)C(OC)=CC(OC)=C2C1=O]

The inventors further discovered that this compound has a high antiproliferative activity, as this was demonstrated on the line of mammary cancer cells MCF-7. Table 2 below describes the comparative antiproliferative activity of 3 active ingredients (tamoxifene, 5-FU and Guieranone B) on the MCF-7 mammary cancer cell line tested under the same conditions in the laboratory. The CI₅₀ observed for Guieranone B (7.09 μM) is comprised between the value observed for tamoxifene and 5-FU. Guieranone B is therefore an interesting active ingredient for the treatment of breast cancers, notably.

TABLE 2

| | Average Cl₅₀ in μg/mL | Average Cl₅₀ in μM |
|---|---|---|
| Tamoxifene | 4.42 ± 0.34 | 11.91 ± 0.93 |
| 5-FU | 0.53 ± 0.05 | 4.04 ± 0.39 |
| Guieranone B | 2.24 ± 0.13 | 7.09 ± 0.41 |

An object of the invention is therefore to propose Guieranone B as a novel compound or pharmaceutical active ingredient, notably as a novel anti-cancer agent.

Another object of the invention is to isolate this novel compound in substantially pure form from Guiera senegalensis.

Another object of the invention is to administer this novel compound to humans or animals for treating cancer.

The characterization of this novel compound and the description of an extraction-purification method give the possibility of having concentrated extracts of this compound available. Notably, the method described in the examples gives the possibility of obtaining a pure compound at more than 89% molar. This measurement was carried out by HPLC. The obtained product may then be used for further purifying the identified and characterized compound, in order to obtain a compound having a higher degree of purity.

The object of the present invention is therefore the compound of formula (I) above, one of its position or structure isomers, or one of its addition salts with pharmaceutically acceptable bases or acids, in isolated form or substantially pure. By substantially pure is meant that the compound (I) is pure at more than 80, 85 or 89% molar.

The object of the invention is also an extract from Guiera senegalensis or a concentrated composition comprising at least 50% molar of the compound of formula (I), notably at least 60, 70, 80, 85 or 89% molar of this compound. The object of the invention is notably a concentrated composition comprising at least 80% molar, preferably at least 85% molar of the compound of formula (I).

According to the invention, this compound or this extract or concentrated composition may be used as a drug, notably as an anti-cancer drug.

The object of the invention is therefore also a pharmaceutical or anti-cancer composition comprising a compound of formula (I), notably substantially pure, or a concentrated composition according to the invention, and a pharmaceutically acceptable carrier or excipient.

Its object is also a composition, notably a pharmaceutical composition, comprising as an active ingredient, an extract according to the invention and a pharmaceutically acceptable carrier or excipient.

The compound according to the invention may be used in combination with at least one other pharmaceutical agent, notably an anti-cancer agent. It may thus be used in combination with other pharmaceutical or anti-cancer agents, authorized by the regulatory authorities.

The object of the invention is therefore also a composition comprising the compound of formula (I), notably substantially pure, or a concentrated composition according to the invention, and (at least) one other pharmaceutical agent or anti-cancer agent, in a pharmaceutically acceptable carrier or excipient.

The object of the invention is also a composition comprising the compound of formula (I), notably substantially pure, or a concentrated composition according to the invention, and (at least) one other pharmaceutical or anti-cancer agent except for an agent present in Guiera senegalensis, in a pharmaceutically acceptable carrier or excipient.

The object of the invention is also a composition comprising the compound of formula (I), notably substantially pure, or a concentrated concentration according to the invention, and (at least) one other pharmaceutical agent or anti-cancer agent, notably except for an agent present in Guiera senegalensis, for simultaneous, separate use or spread out over time, of the compound of formula (I) or of the concentrated composition and of the other pharmaceutical or anti-cancer agent. These other agents appear as a drug which may be administered simultaneously (in a common formulation, optionally an extemporaneous formulation, with the compound of formula (I)), separately (according to a treatment procedure providing concomitant administration of the separate formulations) or further spread out over time (according to a treatment procedure providing administration spread out over time of the separate formulations).

For example, the other agent is selected from tamoxifene, 5-FU, vinorelbin, docetaxel or mixtures thereof. In an embodiment, the other anti-cancer agent is tamoxifene. In another embodiment, it is 5-FU. In another embodiment, it is vinorelbin. In another embodiment, it is docetaxel.

The compound, the extract or the concentrated compositions according to the invention, either associated or not with another agent, may notably be used in the treatment of cancer. In an embodiment, this is breast cancer. In another embodiment, this is prostate cancer. In another embodiment, this is colorectal cancer.

The object of the invention is therefore also the use of the compound of formula (I), notably substantially pure, or a concentrated composition according to the invention, for making a drug, notably a drug for treating cancer. In an embodiment, this is breast cancer, in another embodiment this is prostate cancer. In another embodiment this is colorectal cancer.

The compositions according to the invention may appear in any form suitable for the intended administration route. The parenteral route is the preferential administration route and notably the intravenous route.

The compositions according to the invention for parenteral administration may be aqueous or non-aqueous sterile solutions, suspensions or emulsions. As a solvent or carrier, propylene glycol, vegetable oils, in particular olive oil, and injectable organic esters, for example ethyl oleate may be used. These compositions may also comprise adjuvants in particular wetting, emulsifying or dispersant agents. Sterilization may be accomplished in several ways, for example by means of a bacteriological filter, by incorporating to the composition sterilizing agents, by irradiation or by heating. They may also be in the form of sterile solid compositions which may be dissolved or dispersed in sterile water or in any other injectable sterile medium.

The invention also relates to a method for treating a pathology sensitive to the compound of formula (I), in particular cancer, comprising the administration, to the patient in need thereof, of an effective amount of the compound according to the invention, notably substantially pure or as an extract or a concentrated composition, with a pharmaceutically acceptable carrier or excipient. In an embodiment, this is breast cancer. In another embodiment, this is prostate cancer. In another embodiment this is colorectal cancer.

By sufficient amount is meant an amount of compound according to the present invention effective for limiting development of the disease, causing it to regress or to disappear. The sufficient amount may be determined by one skilled in the art, via a conventional technique and by observing results obtained under similar circumstances. In order to determine the sufficient amount, various factors have to be taken into account by one skilled in the art, notably and without being limited thereto: the subject, his/her size, his/her age, his/her general health condition, the involved disease and its severity level; the response of the patient, the type of compound, the administration mode, the bioavailability of the administered composition, the dosage, the concomitant use of other drugs, etc.

The object of the invention is also the fraction called KP-18^{E}, the preparation method of which is described in the examples. Thus the object of the invention is a pharmaceutical or anticancer composition comprising this fraction, and its uses according to the invention.

The invention will now be described in more detail by means of examples taken as being non-limiting.

### EXAMPLE 1

### Procedure for Extracting the Compound of Formula (I)

Extraction was carried out from 1,100 g of leaves of Guiera senegalensis (harvested near the town of Segue, in the region of Mopti, Mali). The plant material often contains appreciable amounts of fats; it was proceeded with delipidation by mechanical stirring of the milled leaves with 7.5 liters of cyclohexane for 48 hours. The plant material was then filtered in vacuo and then dried.

After drying, the extraction was carried out in 3 steps:

1^{st} Step: The powdered, dilapidated and dried leaves were placed in an Erlenmeyer containing 3.75 liters of methanol. 1.25 liter of a 33% ammonia aqueous solution was added. The solution has a pH comprised between 9 and 10. The whole was stirred mechanically for 48 hours. At the end of the 48 hours, it was proceeded with filtration in vacuo, and the filtrate was then concentrated and 0.65 liter of dichloromethane were added to the filtrate.

2^{nd} Step: The organic phase (dichloromethane) was separated from the marc by means of a decantation funnel. After drying on Na₂SO₄, it was then partly concentrated with the rotary evaporator down to a volume of about 0.4 liter. 0.3 liter of an aqueous solution of hydrochloric acid diluted to 5% was added. This mixture was stirred for 24 hours and the aqueous phase was separated by means of a decantation funnel.

3^{rd} Step: The preceding aqueous phase was alkalinized by adding an aqueous 1M soda solution up to pH=8, in the presence of 0.5 liter of dichloromethane. The organic phase (dichloromethane) was separated by means of a decantation funnel and the aqueous phase was re-extracted twice with 0.2 liter of dichloromethane. The collected organic phases (dichloromethane) were dried on Na₂SO₄, filtered and evaporated under reduced pressure.

3.3 g of a crude extract were thereby obtained.

### EXAMPLE 2

### Procedure for Separating the Crude Extract

Separation by chromatography on silica gel was then carried out. With this step it is possible to separate the molecules according to their polarity. The results of chromatographic tests led to the use as an elution system of a cyclohexane/ethyl acetate/methanol mixture with a polarity gradient.

A mass of about 1 g of crude extract was deposited on a silica gel column (type 60, 230-400 mesh, Merck) prepared in a cyclohexane/ethyl acetate 95/5 mixture. The elution was carried out with a polarity gradient of the cyclohexane/ethyl acetate/methanol system by beginning with a cyclohexane/ethyl acetate 95/5 mixture and by ending with pure methanol for fractionation according to the components. The monitoring of these fractions was carried out by chromatography on a thin layer of silica gel on a glass support. The plates were viewed under UV light (at 254 nm), and then revealed with phosphomolybdic acid. The progression of these columns is gathered in Table 3.

TABLE 3
Results of the separation by chromatography on a column of the
extract of total alkaloids of the leaves from the Segue site.

| | | Elution system | | |
|---|---|---|---|---|
| | No of the | % | % ethyl | % |
| Fraction batch | fraction | cyclohexane | acetate | methanol |
| 01-10 | Sf1 | 95 | 5 | — |
| 11-21 | Sf2 | 95 | 5 | — |
| 22-26 | Sf3 | 90 | 10 | — |
| 27-38 | Sf4 | 90 | 10 | — |
| 39-52 | Sf5 | 90 | 10 | — |
| 53-70 | Sf6 | 90 | 10 | — |
| 71-81 | Sf7 | 90 | 10 | — |
| 82-135 | Sf8 | 85 | 15 | — |
| 139-179 | Sf9 | 85 | 15 | — |
| 180-186 | Sf10 | 80 | 20 | — |
| 187-191 | Sf11 | 80 | 20 | — |
| 192-207 | Sf12 | 80 | 20 | — |
| 208-211 | Sf13 | 80 | 20 | — |
| 212-308 | Sf14 | 75 | 25 | — |
| 309-410 | Sf15 | 75 | 25 | — |
| 411-495 | Sf16 | 65 | 35 | — |
| 496-623 | Sf16 | 55 | 45 | — |
| 624-689 | Sf17 | 40; 30; — | 60; 70; 100 | — |
| 690-696 | Sf18 | — | 90 | 10 |
| 697-734 | Sf19 | — | 80 | 20 |
| 735-755 | Sf20 | — | 50 | 50 |
| — | Sf21 | — | — | 100 |

After gathering them according to the results of the analytic monitoring, the fractions were eluted in a cyclohexane/ethyl acetate/methanol system with different proportions. Thus, it was possible to group certain fractions and to finally obtain 11 fractions for the leaves from Segue (Table 4).

TABLE 4
Grouping the fractions from the chromatographic column of the
extract of total alkaloids of leaves from the Segue site.

| | Name of | |
|---|---|---|
| No. | the fraction | |
| of the | and amount | |
| fractions | (mg) | Observations |
| Sf1-Sf3 | SF1 (38) | 1 compound revealed by phosphomolybdic acid |
| Sf4 | SF2 (3) | Apparently no compound |
| Sf5-Sf6 | SF3 (28) | Mixture of two visible compounds in UV light |
| Sf7 | SF4 (4) | 1 visible compound in UV light |
| Sf8-Sf13 | SF5 (218) | 3 visible compounds in UV light |
| Sf14-Sf16 | SF6 (133) | 2 visible compounds in UV light |
| Sf17 | SF7 (149) | 2 compounds: one visible in UV light and the |
| | | other one revealed by phosphomolybdic acid |
| Sf18 | SF8 (68) | 3 visible compounds in UV light |
| Sf19 | SF9 (122) | as a smear (impurities) |
| Sf20 | SF10 (56) | 3 visible compounds in UV light |
| Sf21 | SF11 (500) | 2 visible compounds in UV light |

### EXAMPLE 3

### Isolation and Purification of the KP-18E Fraction (Guieranone B or Compound of Formula (I))

Chromatographic separation on a column was then carried out on the SF5 fraction which has an interesting anticancer activity. This step allows more accurate separation of the molecules according to their polarity. The result of the chromatographic tests led to the use as an elution system of a cyclohexane/ethyl acetate/dichloromethane/methanol mixture with a polarity gradient.

A mass of 210 mg of SF5 was deposited on a silica gel column (type 60, 230-400 Merck) prepared in cyclohexane/ethyl acetate 90/10. The dimensions of the silica column are a height of 22 cm and a diameter of 3 cm. Elution was achieved by a polarity gradient of the cyclohexane/ethyl acetate/methanol system by beginning with a cyclohexane/ethyl acetate 90/10 mixture and by ending with dichloromethane/methanol (0.2% triethylamine) with fractionation according to the components. The monitoring of these fractions was carried out by chromatography on a thin silica gel layer on a glass support. The plates were viewed under a UV light (at 254 nm), and then revealed with phosphomolybdic acid. The progression of these columns was gathered in the Table 3. After gathering them according to the results of the analytical monitoring, 9 different fractions were obtained.

Among the latter, the KP-19E fraction, representing 17 mg, consists of the compound of the invention which was named Guieranone B.

TABLE 5
Grouping the fractions from the chromatographic
column of fraction SF5.

| | | Name of | |
|---|---|---|---|
| | Frac- | the fraction | |
| Elution system | tion | and amount | |
| (tube numbers) | batch | (mg) | Observations |
| Cyclohex 9/EtOAc 1 | 1-29 | KP-18A | 2 visible spots in UV |
| (1-35) | | (20) | yellow in silica |
| | 30-35 | KP-18B | 1 visible spot in UV and |
| | | (100) | 2 spots revealed with |
| | | | phosphomolybdic acid |
| | | | yellow in silica |
| Cyclohex 8/EtOAc 2 | 36-56 | KP-18C | 3 visible spots in UV |
| (36-52) | | (21) | pink in silica |
| Cyclohex 7/EtOAc 3 | 57-69 | KP-18D | 1 visible spot in UV |
| (53-83) | | (10) | pink in silica |
| Cyclohex 6/EtOAc 4 | 70-86 | KP-18E | 1 visible spot in UV |
| (84-104) | | (17) | pink in silica |
| Cyclohex 5/EtOAc 5 | 87-140 | KP-18F | Visible trace in UV |
| (105-122) | | (9) | pink in silica |
| Cyclohex 4/EtOAc 6 | | | |
| (123-140) | | | |
| Cyclohex 3/EtOAc 7 | | KP-18G | 1 visible spot in UV |
| (100 ml) | | (8) | pink in silica |
| Cyclohex 2/EtOAc 8 | | KP-18H | Visible trace in UV |
| (150 ml) | | (5) | pink in silica |
| Cyclohex 1/EtOAc 9 | | KP-18I | Visible trace in UV |
| EtOAc 100% | | (8) | pink in silica |
| EtOAc 9/MeOH 1 | | | |
| DCM 8/MeOH (0.2 | | | |
| % TEA) 2 | | | |
| (100 ml for each) | | | |

The Guieranone B purity in the SF5 and KP-18E fractions was measured by HPLC on a Symmetry C18 column of size 4.6×150 mm, diameter: 5 mm

mobile phase: gradient [H₂O+trifluoroacetic acid (0.1%)] and MeOH

TABLE 6

| Time | H₂O + trifluoroacetic | |
|---|---|---|
| (min) | acid (0.1%) | MeOH |
| 0 | 95% | 5% |
| 1 | 95% | 5% |
| 45 | 5% | 95% |
| 60 | 95% | 5% |

flow rate: 1 mL/min
detector: UV 254 nm
injected volume: 20 mL

### Results:

SF5: a characteristic peak at 24.062: area under the curve 4.3681% molar
KP-18E: characteristic peak at 24.335: area under the curve 89.1262% molar

The obtaining of a substantially pure compound of formula (I) is demonstrated, with a molar purity of more than 89%.

### EXAMPLE 4

### Complete Characterization of KP-SF5-18^{E} (KP-18E)

Guieranone B (C₁₇H₁₆0₆—316 g·mol⁻¹). TOF (pos.) m/z 339 [M+Na]⁺, HRMS-TOF (pos) m/z 317.10181, ([M+H]⁺, calculated for C₁₇H₁₇O₆, 317.10150).

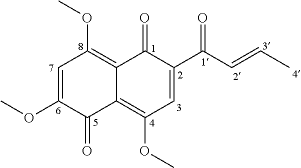 [SMILES: C/C=C/C(=O)C1=CC(OC)=C2C(=O)C(OC)=CC(OC)=C2C1=O]

¹³C NMR (CDCl₃-d1, 100 MHz) δ 18.0 (C4′); 56.0 (OCH₃-4, OCH₃-8); 63.2 (OCH₃-6); 105.5 (C3); 111.9 (C7); 117.3 (C8a); 130.7 (C2); 134.2 (C2′); 134.9 (C4a); 148.9 (C3′); 158.5 (C6); 158.7 (C8); 159.3 (C4); 179.2 (C1); 182.8 (C5); 193.1 (C1′).

¹H NMR (CDCl₃-d1, 400 MHz) δ 7.56 (s, 1H, H3); 6.59 (m, 1H, H3′); 6.30 (d, J=15.5, 1H, H2′); 6.08 (s, 1H, H7); 3.95 (s, 3H, O—CH₃-4); 3.90 (s, 3H, O—CH₃-4); 3.82 (s, 3H, O—CH₃-6); 1.94 (d, J=6.60 Hz, 3H, H4′)

TABLE 7

| Position | H | C | HMBC |
|---|---|---|---|
| 1 | | 179.2 | |
| 2 | | 130.7 | |
| 3 | 7.56, s, 1H | 105.5 | 1, 2, 4, 4a, 8a, 1′, 2′ |
| 4 | | 159.3 | |
| OCH₃-4 | 3.95, s, 3H | 56.0 | 4 |
| 4a | | 134.9 | |
| 5 | | 182.8 | |
| 6 | | 158.5 | |
| OCH₃-6 | 3.82, s, 3H | 63.2 | 6 |
| 7 | 6.08, s, 1H | 111.9 | 1, 5, 6, 8, 8a |
| 8 | | 158.7 | |
| OCH₃-8 | 3.90, s, 3H | 56.0 | 7.8 |
| 8a | | 117.3 | |
| 1′ | | 193.1 | |
| 2′ | 6.30, d, 1H (J = 15.5 Hz) | 134.2 | 1′, 3′, 4′ |
| 3′ | 6.59, m, 1H | 148.9 | 1′, 2′ 4′ |
| 4′ | 1.94, d, 3H (J = 6.60 Hz) | 18.0 | 1′, 2′, 3′ |

The ¹H NMR, COSY (Correlation Spectroscopy), ¹³C and HMBC (¹H/¹³C) spectra show the presence of two aromatic groups and of an ethylenic ketone. Analysis of the ¹H NMR, COSY ¹³C, HSQC and HMBC (¹H/¹³C) spectra allows confirmation of the final structure of KP18E (butanone naphthylmethoxylate).

### EXAMPLE 5

### Detailed Procedure of the Proliferation Measurement of Mammary Cancer Cells MCF-7

### 1^{st} Part Day 1: Sowing the Cells

### 1. Preparation of the Medium:

Preparation of the volume to be put into the plates:

In a 50 mL Falcon, prepare 30 mL of complete RPMI with 10% FCS and 1% Glutamine: 27 mL of RPMI: 3 mL of FCS and 300 μL of Glutamine.

### 2. Preparation of the Cell Suspension:

Suck up the medium contained in the flask with the vacuum pump
Rinse with 3 mL of PBS and suck up with the vacuum pump
Add 1 mL of trypsin and let it act for 3 mins in the oven at 37° C. in order to detach the cells.
When the cells are detached, add 2 mL of complete medium prepared previously for inactivating the trypsin.
Mix well the suspension by suction-discharge and transfer into a well-defined Falcon 15.
Count the cells in a Malassez cell: carry out dilution to one 20^{th} for counting them with Trypan blue.

### 3. Sowing the Plates:

Sow the plates with 200 μL of cell suspension according to the determined scheme
Put 200 μL of water in the peripheral wells
Leave for incubation for 24 hours.

### 2^{nd} Part: Day 2, the Extract is Exposed

### 4. Preparation of the Medium:

A total of 45 mL of medium to be prepared in a Falcon 50 sterilized i.e. 40.5 mL of RPMI+4.5 mL of FCS+450 μL of glutamine

### 5. Preparation of the Extracts:

prepare a 10 mg/mL solution:
Dissolve 8.1 mg of crude extract (noted as SF) according to Example 1, of the SF 5 fraction according to Example 2 or of the KP-18^{E} fraction (Guieranone B) in 810 μL of DMSO
Prepare the mother solution of extract (Ex) at 100 μg/mL i.e. 50 μL of (10 mg/ml) in 4,950 μL of RPMI
Produce the range of extracts (for 1 extract and only 1 range).
Remove the medium in the plates and add 200 μL per well of an extract range (one column per concentration).
Incubate the plates in the oven at 37° C. for 72 hrs.

### 6. Prepare Resazurin (Developer) and Read the Plate to

For T₀:

2 columns of cells+one column of resazurin alone=3 columns

200 μL/well×3 columns×6 lines=3,600 μL (total volume to be prepared: 5 mL)
Prepare 4.9 mL of medium+100 μL resazurin

Remove the medium and add 200 μL of (medium+resazurin) per well.
Incubate for 2 hours and read with fluorskan.
   3^{rd} Part: Measurement of Proliferation at 24 Hours (for t₀), 72 Hrs for Extract
For the extract at 72 hrs:

200 μL/well×18 columns×6 lines (total volume to be prepared: 25 mL)
Prepare 24.5 mL of RPMI+500 μL of resazurin
Remove the medium and add 200 μL of (medium+resazurin) per well.
After 2 hours, readout with fluorskan.

### Results:

1: Measurement of the SF antiproliferative activity in inhibition % (Screening from 0 to 100 μg/mL)

TABLE 8
SF

| | Inhibition percentage | | | | |
|---|---|---|---|---|---|
| | Concentrations | | | | |
| | 0 | 12.5 | 25 | 50 | 100 |
| | μg/mL | μg/mL | μg/mL | μg/mL | μg/mL |
| Values | 0.00 | 98.10 | 96.79 | 100.00 | 100.00 |
| | 11.25 | 98.46 | 97.74 | 100.00 | 100.00 |
| | 19.60 | 98.78 | 97.57 | 100.00 | 100.00 |
| | 0.00 | 98.20 | 98.42 | 100.00 | 100.00 |
| | 5.93 | 97.07 | 97.82 | 100.00 | 100.00 |
| | 5.72 | 98.40 | 96.71 | 100.00 | 100.00 |
| Averages | 7.08 | 98.17 | 97.51 | 100.00 | 100.00 |
| SEM | 3.04 | 0.24 | 0.27 | 0.00 | 0.00 |

2: Measurement of the SF antiproliferative activity in inhibition % (Screening from 0 to 10 μg/mL)

TABLE 9

| SF | Inhibition percentage | | | | | | | |
|---|---|---|---|---|---|---|---|---|
| Concentrations | 0 μg/mL | 0.5 μg/mL | 1 μg/mL | 2 μg/mL | 4 μg/mL | 6 μg/mL | 8 μg/mL | 10 μg/mL |
| Values | 0.00 | 0.95 | 5.83 | 28.14 | 57.42 | 78.82 | 96.93 | 96.63 |
| | 0.00 | 10.85 | 26.32 | 40.97 | 73.14 | 87.61 | 98.56 | 97.98 |
| | 13.68 | 28.37 | 30.18 | 45.99 | 76.24 | 91.83 | 98.86 | 97.89 |
| Averages | 4.56 | 13.39 | 20.78 | 38.36 | 68.94 | 86.09 | 98.12 | 97.50 |
| SEM | 4.56 | 8.02 | 7.56 | 5.31 | 5.83 | 3.83 | 0.60 | 0.44 |

3: Measurement of the SF antiproliferative activity in inhibition %

(Averages obtained for the 6 tests)

TABLE 10

| SF | | | 0 μg/mL | 0.5 μg/mL | 1 μg/mL | 2 μg/mL | 3 μg/mL | 4 μg/mL | 6 μg/mL | 8 μg/mL |
|---|---|---|---|---|---|---|---|---|---|---|
| 1 | N1 | Averages | 1.87 | 0.00 | 0.00 | 9.54 | 32.14 | 58.48 | 85.92 | 97.97 |
| | N2 | Averages | 1.81 | 0.00 | 2.25 | 9.19 | 14.19 | 34.87 | 67.82 | 96.44 |
| | N3 | Averages | 1.43 | 0.00 | 0.00 | 6.34 | 17.06 | 42.39 | 74.72 | 97.54 |
| 2 | N1 | Averages | 0.74 | 4.73 | 5.34 | 16.93 | 35.35 | 56.34 | 81.25 | 95.94 |
| | N2 | Averages | 0.89 | 3.68 | 2.74 | 21.04 | 43.86 | 62.87 | 85.48 | 96.05 |
| | N3 | Averages | 1.50 | 3.20 | 1.13 | 8.84 | 13.44 | 36.69 | 62.80 | 94.15 |

| | | | | | | | | |
|---|---|---|---|---|---|---|---|---|
| Average of the | 1.37 | 1.93 | 1.91 | 11.98 | 26.00 | 48.60 | 76.33 | 96.34 |
| experiments | | | | | | | | |
| SEM | 0.19 | 0.88 | 0.82 | 2.32 | 5.23 | 4.93 | 3.90 | 0.55 |

CI50

| | | | |
|---|---|---|---|
| 1 | N1 | 3.64 | μg/mL |
| | N2 | 4.81 | |
| | N3 | 4.40 | |
| 2 | N1 | 3.66 | |
| | N2 | 3.29 | |
| | N3 | 4.91 | |

| | | |
|---|---|---|
| Average | 4.12 ± 0.27 | μg/mL |
| CI50 ± SEM | | |

4: Inhibition percentage of the proliferation of cancer cells MCF-7 versus SF5 concentrations of the screening

TABLE 11

| SF5 | Inhibition percentage | | | | | | | |
|---|---|---|---|---|---|---|---|---|
| Concentrations | 0 μg/mL | 0.5 μg/mL | 1 μg/mL | 2 μg/mL | 3 μg/mL | 4 μg/mL | 6 μg/mL | 8 μg/mL |
| Values | 3.55 | 0 | 10.52 | 32.23 | 54.69 | 62.03 | 72.34 | 92.45 |
| | 0 | 0 | 3.52 | 27.79 | 52.88 | 58.91 | 73.28 | 94.76 |
| | 2.24 | 0 | 5.96 | 35.51 | 57.75 | 63.19 | 75.16 | 92.31 |
| | 0 | 0 | 2.27 | 33.29 | 52.72 | 59.78 | 68.72 | 90.60 |
| | 1.58 | 0 | 0 | 34.57 | 56.41 | 60.19 | 72.65 | 89.37 |
| | 1.30 | 0 | 3.46 | 31.26 | 55.16 | 60.06 | 73.06 | 87.83 |
| Averages | 1.44 | 0 | 4.29 | 32.44 | 54.94 | 60.69 | 72.53 | 91.22 |
| SEM | 0.55 | 0 | 1.47 | 1.12 | 0.80 | 0.65 | 0.86 | 1.00 |

5: Measurement of SF:SF5 fraction antiproliferative activity in inhibition % (Averages obtained for the 6 tests)

TABLE 12

| SF5 | | | 0 μg/mL | 0.5 μg/mL | 1 μg/mL | 2 μg/mL | 3 μg/mL | 4 μg/mL | 6 μg/mL | 8 μg/mL |
|---|---|---|---|---|---|---|---|---|---|---|
| 1 | N1 | Averages | 2.47 | 1.02 | 26.74 | 74.39 | 84.77 | 86.41 | 89.95 | 94.46 |
| | N2 | Averages | 1.22 | 0.71 | 21.56 | 72.14 | 82.94 | 86.30 | 90.50 | 94.89 |
| | N3 | Averages | 1.62 | 6.45 | 17.76 | 70.17 | 82.82 | 86.00 | 90.15 | 94.31 |
| 2 | N1 | Averages | 3.14 | 18.51 | 55.12 | 84.52 | 92.33 | 95.50 | 99.30 | 100.00 |
| | N2 | Averages | 1.76 | 19.97 | 47.68 | 79.40 | 89.05 | 94.02 | 97.60 | 100.00 |
| | N3 | Averages | 2.07 | 20.15 | 50.33 | 82.44 | 90.30 | 92.81 | 95.08 | 98.87 |

| | | | | | | | | |
|---|---|---|---|---|---|---|---|---|
| Average of the | 2.04 | 11.13 | 36.53 | 77.17 | 87.03 | 90.17 | 93.76 | 97.09 |
| experiments | | | | | | | | |
| SEM | 0.27 | 3.85 | 6.66 | 2.37 | 1.65 | 1.79 | 1.68 | 1.14 |

CI50 SF5

| | | | |
|---|---|---|---|
| 1 | N1 | 1.40 | μg/mL |
| | N2 | 1.47 | |
| | N3 | 1.53 | |
| 2 | N1 | 0.90 | |
| | N2 | 1.05 | |
| | N3 | 0.99 | |

| | | |
|---|---|---|
| Average | 1.22 ± 0.11 | μg/mL |
| C150 ± SEM | | |

6: Inhibition percentage of the proliferation of cancer cells MCF-7 versus the concentrations of KP-18E of the screening

TABLE 13

| KP-18E | Inhibition percentage | | | | | | | | | |
|---|---|---|---|---|---|---|---|---|---|---|
| Concentrations | 0 μg/mL | 0.25 μg/mL | 0.5 μg/mL | 1 μg/mL | 1.5 μg/mL | 2 μg/mL | 2.5 μg/mL | 3 μg/mL | 4 μg/mL | 5 μg/mL |
| Values | 30.82 | 0.00 | 1.98 | 15.52 | 36.10 | 62.54 | 57.84 | 77.40 | 99.38 | 100.00 |
| | 0.00 | 0.00 | 0.00 | 0.00 | 48.15 | 43.35 | 59.61 | 86.16 | 100.00 | 100.00 |
| | 0.00 | 0.00 | 7.74 | 29.28 | 50.66 | 59.58 | 65.92 | 83.19 | 100.00 | 100.00 |
| | 22.56 | 0.00 | 22.99 | 27.15 | 53.59 | 61.13 | 65.73 | 84.81 | 100.00 | 100.00 |
| | 0.00 | 0.00 | 1.87 | 39.25 | 53.16 | 48.79 | 68.02 | 92.10 | 100.00 | 100.00 |
| | 3.37 | 25.60 | 31.52 | 37.44 | 49.86 | 68.59 | 69.73 | 94.22 | 100.00 | 100.00 |
| Averages | 9.46 | 4.27 | 11.02 | 24.77 | 48.59 | 57.33 | 64.48 | 86.31 | 99.90 | 100.00 |
| SEM | 5.58 | 4.27 | 5.36 | 6.04 | 2.63 | 3.84 | 1.93 | 2.50 | 0.10 | 0.00 |

7: Measurement of the KP-18E antiproliferative activity in inhibition % (Averages obtained for the 6 tests)

TABLE 14

| | | | μg/mL | | | | | | | | | |
|---|---|---|---|---|---|---|---|---|---|---|---|---|
| | | | 0 | 0.25 | 0.5 | 1 | 1.5 | 2 | 2.5 | 3 | 4 | 5 |
| 1 | N1 | Averages | 3.23 | 0.89 | 4.08 | 16.32 | 33.96 | 46.74 | 74.28 | 95.88 | 99.55 | 99.43 |
| | N2 | Averages | 1.95 | 0.00 | 0.30 | 6.50 | 8.86 | 23.88 | 40.33 | 61.22 | 98.27 | 99.91 |
| | N3 | Averages | 3.22 | 0.53 | 2.19 | 2.87 | 22.68 | 41.08 | 65.99 | 93.83 | 99.46 | 99.29 |
| 2 | N1 | Averages | 1.79 | 1.75 | 3.42 | 15.89 | 20.44 | 38.75 | 49.01 | 57.82 | 91.14 | 99.02 |
| | N2 | Averages | 1.90 | 4.00 | 7.57 | 20.89 | 32.06 | 48.73 | 57.31 | 70.40 | 99.55 | 100.00 |
| | N3 | Averages | 3.78 | 7.53 | 10.24 | 28.50 | 40.50 | 51.63 | 61.79 | 75.79 | 99.83 | 100.00 |

| | | | | | | | | | | |
|---|---|---|---|---|---|---|---|---|---|---|
| Average of the | 2.65 | 2.45 | 4.63 | 15.16 | 26.41 | 41.80 | 58.12 | 75.83 | 97.96 | 99.61 |
| experiments | | | | | | | | | | |
| SEM | 0.35 | 1.17 | 1.49 | 3.83 | 4.64 | 4.08 | 4.96 | 6.57 | 1.38 | 0.17 |

CI50

| | | | |
|---|---|---|---|
| 1 | N1 | 2.0535 | μg/mL |
| | N2 | 2.7202 | |
| | N3 | 2.1664 | |
| 2 | N1 | 2.5518 | μg/mL |
| | N2 | 2.067 | |
| | N3 | 1.9176 | |

| | | |
|---|---|---|
| Average C150 | 2.246083333 | μg/mL |
| SEM | 0.1293 | μg/mL |

### EXAMPLE 6

### Comparison of the Antiproliferative Activity of Guieranone B with that of Tamoxifene and 5-FU

The procedure described in Example 5 was used by replacing Guieranone B with tamoxifene or 5-FU (5-Fluorouracil). By following this procedure, it was possible to measure the CI₅₀s of these compounds and of comparing them with that of Guieranone B.

Table 15 (identical with Table 2) describes the compared antiproliferative activity of these 3 active ingredients (tamoxifene, 5-Fu and Guieranone B) on the mammary cancer line MCF-7 tested under the same conditions.

| | Average CI₅₀ in μg/mL | Average CI₅₀ in μM |
|---|---|---|
| Tamoxifene | 4.42 ± 0.34 | 11.91 ± 0.93 |
| 5-FU | 0.53 ± 0.05 | 4.04 ± 0.39 |
| Guieranone B | 2.24 ± 0.13 | 7.09 ± 0.41 |

The CI₅₀ observed for Guieranone B (7.09 μM) is comprised between the values observed for tamoxifene and 5-FU. Guieranone B is therefore a potentially interesting active ingredient for treating breast cancers.

1. A substantially pure compound of formula (I) 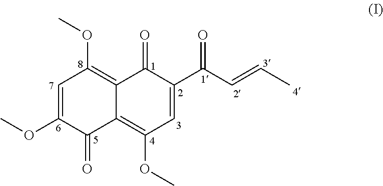 [SMILES: C/C=C/C(=O)C1=CC(OC)=C2C(=O)C(OC)=CC(OC)=C2C1=O]
one of its position or structure isomers, or one of its addition salts with pharmaceutically acceptable bases or acids.

2. A concentrated composition comprising at least 80% molar, preferably at least 85% molar, of the compound of formula (I) according to claim 1.

3. A pharmaceutical or anticancer composition comprising a compound according to claim 1 or a concentrated composition according to claim 2 and at least one pharmaceutically acceptable carrier or excipient.

4. The pharmaceutical or anticancer composition according to claim 3, comprising at least one other pharmaceutical or anticancer agent.

5. The pharmaceutical or anticancer composition according to claim 4, for simultaneous, separate use or spread out over time of the compound of formula (I) or of the concentrated composition according to claim 2 and of the other pharmaceutical or anticancer agent.

6. The pharmaceutical or anticancer composition according to claim 3 or 4 wherein the other agent is selected from tamoxifene, 5-FU, vinorelbin, docetaxel or mixtures thereof.

7. The compound according to claim 1, the concentrated composition according to claim 2 or the pharmaceutical or anticancer composition according to claims 3 to 6 as a drug, preferably as an anticancer drug.

8. The compound according to claim 1, the concentrated composition according to claim 2 or the pharmaceutical or anticancer composition according to claims 3 to 6, for its use in the treatment of cancer, preferably for treating breast cancer, colorectal cancer or prostate cancer.

9. An extract of Guiera senegalensis comprising more than 50% molar of the compound of claim 1, as an anticancer drug, preferably for treating breast cancer, colorectal cancer or prostate cancer.

10. An extract of Guiera senegalensis comprising more than 50% molar of the compound of claim 1, as an anticancer drug, preferably to treating breast cancer, colorectal cancer or prostate cancer.
